SEQUENCE LISTING (4 sequences)

SEQ ID NO: 1 - protein, 1319 aa, Salmonid alphavirus
MFPMQFTNSAYRQMEPMFAPGPRGQVQPYRPRTKRRQEPQVGNAAIAALANQMSALQLQVAGLAGQARVDRRGPRRVQKSKQKKKNPSNGEKPKEKKKKQKQQEKKGSGGVIKKPRNRPGKEVRISVKRARQSTFPVYHDGAISGYAVLIGSRVFKPAHVKGKIDHPELADIKFQVAEDMDLEAAAYPKSMRDQAAEPATMMDGVYNWEYGTIRVEDNVVIDASGRGKPGDSGRAITDNSGKVVGIVLGGGPDGRRTRLSVIGFDKKMKAREIAYSEAIPWTRAPALLLLPMVIACTYNSNTFDCSKPSCQDCCITAEPEKAMAMLKDNLNDPNYWDLLIAVTTCNSARKKRAVSASPAAVYDTQILAAHAAASPYRAYCPDCDGTACISPIAIDEVVSSGSDHVLRIRVGSQSGVTAKGGAAGETSLRYLGRDGKVHAADDTRLVVRTTAKCDVLQATGHYILASCPEGQSITVAATLDGTRHQCTTVFEHQVTEKFTRERSKGHHLSDLTKKCTRFSTTPKKSAPYLVDVYDALPISVEISTVVTCNDNQCTVRVSPGTTVKFDKKCKSAAQATVTFTSDSQTFTCEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELTWGNNAPLHFWSSVRYASGDADAYPWELLVHHIKHHPEYAWAFVGVACGLLVIAVCMFACACNRVRYSLVANTFNPNPPPLTALTAALCCIPGARADQPYLDIIAYLWTNSKVAFGLQCAAPVACVLIVTYALRHCRLCCKSFLGVRGWSALLVILAYVQSCKSYEHTVVVPMDPRAPSYEAVINRNGYDPLKLTIAVNFTVISPTTALEYWTCASVPIVEPPHVGCCTSVSCPSDLSTLHAFTGKAVSDVHCDVHTNVYPLLWGAAHCFCSTENTQVSAVAATVSEFCAQDSERAEAFSVHSSSVTAEVLVTLGEVVTAVHVYVDGVTSARGTDLKIVAGPITTDYSPFDRKVVRIGEEVYNYDWPPYGAGRPGTFGDIQARSTNYVKPNDLYGDIGIEVLQPTNDHVHVAYTYTTSGLLRWLQDAPKPLSVTAPHGCKISANPLLALDCGVGAVPMSINIPDAKFTRKLKDPKPSALKCVVDSCEYGVDYGGAATITYEGHEAGKCGIHSLTPGVPLRTSVVEVVAGANTVKTTFSSPTPEVTLEVEICSAMVTCASKCTPPKEHVVATRPRHGSDTGGYISGPAMRWAGGIVGTLAVLFLILAVTYCVVKKCRSKRIRIVKS

SEQ ID NO: 2 - protein, 1320 aa, Salmonid alphavirus
MFPMQFTNSAYRQMEPMFAPASRGQVQPYRPRTKRRQEPQVGNAAIAALANQMSALQLQVAGLAGQARVDRRGPRRVQKNKQKKKNSSNGEKPKEKKKKQKQQEKKGSGGEKVKKPRNRPGKEVRISVKRARQSTFPVYHDGAISGYAVLIGSRVFKPAHVKGKIDHPELADIKFQVAEDMDLEAAAYPKSMRDQAAEPATMTDGVYNWEYGTIRVEDNVVIDASGRGKPGDSGRAITDNSGKVVGIVLGGGPDGRRTRLSVIGFDKKLKAREIAYSEAIPWTRAPALLLLPMVIACTYNSNTFDCSKPSCQDCCITAEPKKAMTMLKDNLNDPNYWDLLIAVTTCSSARKKRAVSTSPAAAYDTQILAAHAAASPYRAYCPDCDGTACISPIAIDEVVSSGSDHVLRIRVGSQSGVTAKGGAAGETSLRYLGRDGKVHAADNTRLVVRTTAKCDVLQATGHYILANCPVGQSLTVAATLDGTRHQCTTVFEHQVTEKFTRERSKGHHLSDLTKKCTRFSTTPKKSALYLVDVYDALPISVEISTVVTCNESQCTVRVPPGTTVKFDKKCKSAAQATVTFTSGSQTFTCEEPVLTAASITQGKPHLRSSMLPSGGKEVKARIPFPFPPETATCRVSVAPLPSITYEESDVLLAGTAKYPVLLTTRNLGFHSNATSEWIQGKYLRRIPVTPQGIELMWGNNAPLHFWSSVRYASGDADAYPWELLVHHIKHHPEYAWAFVGVACGLLAVAACVFACACNRVRYSLLANTFNPNPPPLTALTAALCCIPGARADQPYLDIIAYLWTNSKVAFGLQCAAPVACMLIVTYALRHCRLCCKSFLGVRGWSALLVILAYVQSCKSYEHTVVVPMDPRAPSYEAVINRNGYDPLKLTIAVNFTVISPTTALEYWTCAGVPVVEPPHVGCCTSVSCPTDLSTLHAFTGKAVSDVHCDVHTNVYPLLWGAAHCFCSTENTQVSAVAATVSEFCAQDAERAEAFSVHSSSVTAEILVTLGEVVTAVHVYVDGVTSARGTDLKIVAGPITTDYSPFDRKVVRISEEVYNYDWPPYGAGRPGTFGDIQARSTNYVKPNDLYGDIGIEVLQPTNDHVHVAYTYTTSGLLRWLQDAPKPLSVTAPHGCKISANPLLALDCGVGAVPMSINIPDAKFTRKLKDPKPSALKCVVDSCEYGVDYGGAATITYEGHEAGKCGIHSLTPGVPLRTSVVEVVAGANTVKTTFSSPTPEVTLEVEICSAIVKCASECTPPKEHVVAARPRHGSDTGGYISGPAMRWAGGIVGTLVVLFLILAVTYCVVKKCRSKRIRIVKS

SEQ ID NO: 3 - protein, 861 aa, Piscine myocarditis virus
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRYDNQARAAQVALCRERADMAECPWGGYRERGVRPGSVANWQYVRFDPTVAVGVAAHFWSVVKVMVAPVPDRAAALADMAWGKGKVQAMGEDVINGQMGQPESMMRGVALNENQGLAAATVRRVVGLENESMQTTHWSTTEVAMNGYYGRAGATAHHAAFPLSEGGTMRKRIPAIEMRENGVEGDLMNDDLYSIGTAAGYLAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

SEQ ID NO: 4 - protein, 666 aa, synthetic construct
  source 1..666: note = recombinant Piscine myocarditis virus antigen
MEPNTSVIATEQQQAAMREVEAEAAARDEVVEKIAFAEGAMMVQTRRLPSGKSSVGGFLGELAQNIRAMNRSLHTDTNMLTEGAMVDRARAKVHKIIREGNLDSRVFSNTGSNTMLSLWVPAVPGPPAVPEHWDVAPSWFVCRPGKKGGIKITQSASMAALNPLFRGADVGPIGTAVRADVNAFSMNAVLGALRAGGFNTEHSLVSFVEPLIRILLMGVQTQDRGTSPWDWVGGMSSRIVNPLVFTTSGNFFPGGPNLRVWGANDTVARIVNVEDYMREAAGEGRFDAGWGPEFWGGTGDDAVAVVPIRAVEAGLGEVNAGWTLAHMEYPVKVRLLDVDDRTIGPGGSLPLNANREYTAAGATHVPGPYARVLYVVVDQNADRCVGVRVQGQGAVIDVDPALNYVIGGADLGMLPLIQWSVGLGAEDMAQGSIAQTQRWVRMYGNEDDWESAWHLVSSAYTVYSPAFRRSGVAVEGGFWAQPAAGAAPFPLGGLAGWVRGGGGSAVEGMAGAQGGIWDVVQYQLPGPDDEARGVMNTVGAMGGWTRAVTPVDNVATMRDNGVEGEPCGIVMSLPTSGTAVVDRLANFGLPPARAELREVPFGGYQRSVTNTNHRVKVSVSGGRAVVQKGNKAEMNPVFVNRTPGQTTLGQPTTDTTGMTTADFLDI

(19) United States
(12) Patent Application Publication
(10) Pub. No.: US 2026/0053907 A1
(43) Pub. Date: Feb. 26, 2026

(54) METHOD OF ADMINISTRATION OF AQUACULTURE VACCINES

(71) Applicant: Zoetis Services LLC, Parsippany, NJ (US)

(72) Inventors: Espen HAALAND, Baerums Verk (NO); Øyvind HAUGLAND, Oslo (NO); Marius Andre de Feijter KARLSEN, Oslo (NO)

(21) Appl. No.: 19/110,860

(22) PCT Filed: Sep. 11, 2023
(86) PCT No.: PCT/US2023/073825

§ 371 Date: Mar. 12, 2025

Related U.S. Application Data
(60) Provisional application No. 63/375,285, filed on Sep. 12, 2022.

(51) Int. Cl. A61K 39/12; A61K 39/00; A61P 37/04

(52) U.S. Cl. CPC A61K 39/12; A61P 37/04; A61K 2039/53; A61K 2039/545; A61K 2039/552

(57) ABSTRACT

A method of delivering fish DNA vaccines is provided, wherein the vaccines are delivered intramuscularly in a plurality of injections.

### FIELD OF THE INVENTION

This invention is generally in the field of DNA vaccines for aquaculture.

### BACKGROUND

Aquaculture has experienced an enormous growth in productive terms, accounting to >527% in the 1990-2018 time frame. In 2018, aquaculture contributed to approximately 46% of the global total production of aquatic organisms (179 M tons) and 52% of seafood for human consumption (fish, crustaceans, mollusks, and other aquatic animals, excluding aquatic mammals, reptiles, seaweeds, and other aquatic plants).

Commercial aquaculture is impacted by infectious diseases caused primarily by bacteria, viruses, parasites, and, to a lesser extent, fungi. Bacterial diseases can inflict significant biological, thus economic losses. While these are usually controllable with antibiotics, the indiscriminate use of these pharmaceuticals is ultimately a threat to human health because of the development and transfer of resistance mechanisms among bacterial species, some of which are also human pathogens. In addition, some traditional vaccines based on inactivated viruses may cause unacceptable side effect and/or some pathogens are difficult to grow in culture.

Pancreas disease (PD) has represented an economically important viral disease affecting seawater-reared Atlantic salmon (Salmo salar) in Ireland, UK and Norway. The aetiological agent is formally named salmon pancreas disease virus (SPDV), but commonly denoted as salmonid alphavirus (SAV) partly because the virus also infects other salmonid species. Oil-adjuvanted vaccines containing inactivated whole viral particles of SPDV have been used for more than a decade to reduce the impact of PD for the salmonid aquaculture industry. However, in some cases, the use of these vaccines has led to considerable side effects in the form of deformities of the vertebral column and fibrosis in the surrounding musculature, resulting in downgrading at slaughter, reduced growth and animal welfare concerns. See Bæverfjord et al, NOFIMA report 35\2021; and Holm et al, Aquaculture, 526, 2020.

Cardiomyopathy Syndrome (CMS) is an inflammatory heart disease, primarily affecting farmed Atlantic salmon, Salmo salar L. The disease was first detected in Norway in 1985 and has since been diagnosed in both farmed and wild Atlantic salmon. The causative agent of CMS has been identified as piscine myocarditis virus (PMCV) and the isolation and characterization of the virus is described in WO 2011/131600. Methods of controlling the virus are urgently required but despite intensive research, it has not been possible to efficiently culture PMCV, or consequently to produce vaccines based on the attenuated or inactivated virus.

DNA vaccines have become an attractive approach for generating antigen-specific immune responses because of their stability and simplicity of delivery. DNA vaccines can be easily prepared in large scale with high purity, repeatedly administered and are highly stable relative to proteins and other biological polymers.

Among the many forms of nucleic acid vaccine that can be constructed, circular DNA plasmids are the simplest. DNA vaccination involves immunization with a circular DNA plasmid that contains the gene (or genes) that code for an antigen. Indeed, injection of free DNA (naked DNA) stimulates effective and long-time immune responses to the protein (antigen) encoded by the gene vaccine. When plasmid DNA is injected into an individual, the plasmid is taken up by cells and its genetic information is translated into the immunizing protein. This enables the host immune system to respond to the antigen.

However, DNA vaccines are relatively new, and need exists in improving the efficiency of these vaccines.

### SUMMARY OF INVENTION

In one aspect, the disclosure provides a vaccine comprising a nucleic acid sequence encoding an antigen derived from a pathogen affecting fish, for use in a method to actively protect fish against the pathogen, said method comprising administering said vaccine to said fish intramuscularly in a plurality of injections, wherein
a. members of the plurality are delivered into different injection sites;
b. each member of said plurality contains less than an effective dose of said vaccine; and
c. the total dose of said vaccine is no greater than the effective dose; and
d. all members of the plurality are delivered to said fish substantially simultaneously.

Also disclosed are the vaccines as described above wherein the members of the plurality act synergistically.

The vaccine according to claim 1 or claim 2, wherein the total dose provides a faster onset of immunity than the effective dose administered in a single injection, when administered to fish kept under the same conditions.

In certain aspects of the vaccine practiced according to the methods described herein, the total dose of said vaccine is less than the effective dose.

In another aspect, the disclosure provides a vaccine comprising a nucleic acid sequence encoding an antigen derived from a pathogen affecting fish, for use in a method to actively protect fish against the pathogen, said method comprising administering said vaccine to said fish intramuscularly in a plurality of injections, wherein
a. the members of the plurality are delivered into different injection sites;
b. all members of the plurality are delivered to said fish substantially simultaneously, and
   wherein the members of the plurality act synergistically or wherein the total dose administered in the plurality of the injections provides a faster onset of immunity than the same dose administered in a single injection of the same formulation, when administered to fish kept under the same conditions. Preferably, the members of the plurality act synergistically and the total dose administered in the plurality of the injections provides a faster onset of immunity than the same dose administered in a single injection of the same formulation, when administered to fish kept under the same conditions.

Preferably the plurality has 2, 3, or 4 members. The plurality can be delivered within 60 seconds, or more preferably, within 30 seconds, or even more preferably, within 15 seconds.

Also disclosed is a vaccine as described above, wherein the fish is a salmonid and the pathogen is selected from the group consisting of salmonid alphavirus (SAV), viral hemorrhagic septicemia virus (VHSV) Infectious hematopoietic necrosis virus (INHV), infectious pancreatic necrosis virus (IPNV), Infectious salmon anaemia (ISA) virus (ISAV), Piscine Myocarditis virus (PMCV), and Piscine Orthoreovirus (PRV).

In certain embodiments of the vaccine described above, the pathogen is salmonid alphavirus and the antigen comprises SEQ ID NO: 1 or SEQ ID NO: 2. In certain embodiments, the pathogen is PMCV and the antigen comprises SEQ ID NO: 3 or SEQ ID NO: 4. In certain embodiments, the vaccine comprises the antigens both against salmonid alphavirus and against PMCV.

Also disclosed is a vaccine as described above, therein the fish is tilapia and the pathogen is selected from the group consisting of Viral Nervous Necrosis Virus (NNV), Infectious Spleen and Kidney Necrosis Virus (ISKNV), and Tilapia Lake Virus.

Also disclosed are the vaccines as described above, wherein the nucleic acid sequence encoding said antigen is delivered by a heterologous vector, such as a plasmid vector or a viral vector.

Also disclosed are the vaccines as described above, wherein the heterologous vector further comprises a nucleic acid sequence encoding a molecular immunomodulator. In certain embodiments, the molecular immunomodulator is an interferon.

Also disclosed are the vaccines as described above, further comprising an adjuvant.

Also disclosed are the vaccines as described above, wherein said vaccine comprises a means for transporting said nucleic-acid-based vaccine across cell membrane. In certain embodiments, the means comprise a lipid coating.

In another aspect, the disclosure provides an article of manufacturing comprising:
a) a reservoir containing the vaccine according to any one of claims 1-20, said reservoir operably connected to a plurality of needles, said plurality of needles configured to deliver the members of the plurality of injections into different injection sites; and
b) Means for applying pressure to said reservoir thereby pushing the vaccine through the plurality of needles.

In yet another aspect, the disclosure provides article of manufacturing comprising:
a. a plurality of reservoirs, each reservoir containing a member of the plurality of injections of the vaccine according to any one of claims 1-21, wherein each reservoir is operably connected to a needle, wherein needles connected to different reservoirs are configured to deliver the members of the plurality of injection into different injection sites;
b. one or more means for applying pressure to said reservoirs thereby pushing the vaccine through the plurality of needles.

In both this and the previous aspect of the invention, the means may be derived from multiple sources including without limitations, pneumatic, hydraulic, electrical or mechanical sources.

In yet another aspect, the disclosure provides a method of administering an effective dose of a nucleic-acid-based vaccine to a fish, the method comprising delivering a plurality of portions of said effective dose into different injection sites of said fish, wherein said plurality of portions is injected substantially simultaneously.

In certain embodiments, in the method disclosed herein, the plurality has 2, 3, or 4 members and can be delivered within 60 seconds, or 30 seconds, or 15 seconds. In certain embodiment, the plurality has 2 members and is delivered in 15 seconds or less. In other embodiments, the plurality has three members and is delivered in 15 seconds or less.

In certain embodiments of the method described in this aspect, the fish is a salmonid and the pathogen is selected from the group consisting of salmonid alphavirus (SAV), viral hemorrhagic septicemia virus (VHSV) Infectious hematopoietic necrosis virus (INHV), infectious pancreatic necrosis virus (IPNV), Infectious salmon anaemia (ISA) virus (ISAV), Piscine Myocarditis virus (PMCV), and Piscine Orthoreovirus (PRV).

In certain embodiments of the method of this aspect of the invention, the pathogen is salmonid alphavirus and the antigen comprises SEQ ID NO: 1 or SEQ ID NO: 2. In certain embodiments, the pathogen is PMCV and the antigen comprises SEQ ID NO: 3 or SEQ ID NO: 4.

Also disclosed is the method according to this aspect of the invention, wherein the fish is tilapia and the pathogen is selected from the group consisting of Viral Nervous Necrosis Virus (NNV), Infectious Spleen and Kidney Necrosis Virus (ISKNV), and Tilapia Lake Virus.

In certain embodiments of the method according to this aspect of the invention, said nucleic acid sequence encoding said antigen is delivered by a heterologous vector. In certain embodiments, the heterologous vector is a plasmid vector. In other embodiments, the heterologous vector is a viral vector.

Also disclosed is the method according to this aspect of the invention, wherein the heterologous vector further comprises a nucleic acid sequence encoding a molecular immunomodulator. In certain embodiments, the molecular immunomodulator is an interferon.

Also disclosed is the method according to this aspect of the invention, wherein the vaccines further comprise an adjuvant.

Also disclosed is the method according to this aspect of the invention, wherein said vaccine comprises a means for transporting said nucleic-acid-based vaccine across cell membrane. In certain embodiments, the means comprise a lipid coating.

Also disclosed is the method according to this aspect of the invention, wherein the members of the plurality of portions act synergistically.

Also disclosed is the method according to this aspect of the invention, wherein the total dose provides a faster onset of immunity than the effective dose administered in a single injection.

### DETAILED DESCRIPTION

For a better understanding the invention, the following definitions are provided

The term “about” as applied to a reference number refers to the reference number plus or minus 10 percent of said value.

The term “different injection sites” refers to injection points located sufficiently apart from each other so that different skin penetrations are made for the injections into these injection points. For example, the injection sites may be at least 0.5 mm apart. In different embodiments, the injection sites are 0.5 to about 100 mm apart or 0.5 to about 50 mm apart from each other, or about 1 to about 40 mm apart, or about 3 to about 30 mm apart or about 5 to about 25 mm apart, or about 5 to about 20 mm apart, or about 10 to about 20 mm apart, or about 10 mm to about 20 mm apart, or about 10 mm to about 20 mm apart, or about 10 mm to about 30 mm apart, or about 10 mm to about 40 mm apart, or about 10 mm to about 50 mm apart, or about 10 mm to about 60 mm apart, or about 10 mm to about 70 mm apart, or about 10 mm to about 80 mm apart, or about 10 mm to about 90 mm apart, or about 20 mm to about 30 mm apart, or about 20 mm to about 40 mm apart, or about 20 mm to about 50 mm apart, or about 20 mm to about 70 mm apart, or about 30 mm to about 40 mm apart.

The term “effective dose” refers to the amount of the antigen which, if administered as a single injection in a given formulation, would provide the desired level protection against the intended pathogen.

The term “members of the plurality acting synergistically” refers to
a) the ability of the total dose to evoke a greater protective immune response than the same formulation containing the effective dose if the total dose is substantially equal to the effective dose; or
b) the ability of the total dose to evoke the same protective immune response than the same formulation containing the effective dose if the total dose is less than the effective dose.

The term “nucleic-acid-based vaccine” refers to a vaccine where the antigen is encoded by a nucleic acid sequence. The antigen-encoding nucleic acid sequence needs to enter the cell, replicate, and be expressed into the antigen. Nucleic-acid-based vaccines include, without limitation DNA vaccines and mRNA vaccines. DNA vaccines may be vectored by a plasmid or by a heterologous viral vector.

The term “substantially simultaneously” refers to time interval between the injections of the portions of the effective dose of the vaccine disclosed herein. The portions are injected substantially simultaneously if the last portion of the vaccine is injected no later than 5 minutes after the first portion, preferably, no later than 4 minutes, no later than 3 minutes, no later than 2 minutes, no later than 1 minute, no later than 45 seconds, no later than 30 second, and no later than 15 seconds, and no later than 5 seconds, and no later than 1 second.

The term “substantially the same amount of the antigen” in the members of the plurality of injections refers to the amount of the antigen in each member of the plurality of N injections that varies between 1/2N to 1.5/N of the total dose of the antigen. Thus, if there are two members of the plurality of injections, each member of the plurality should preferably contain from 1/4 of the total dose to ¾ of the total dose. If there are three members of the plurality, each member of the plurality should preferably contain from 1/6 to ½ of the total dose. In the most preferred embodiment, each member of the plurality of N injection should contain about 1/Nth of the total dose of the antigen. Thus, if N=2 (two members of the plurality of injections), each member of the plurality should contain about ½ of the total dose. If N=3 (three members of the plurality of the injections), each member of the plurality should contain about ⅓ of the total dose.

The expression “to protect against a pathogen” refers both to the lack (or decreased level) of infection and the lack (or decreased level) of symptoms associated with the infection. If a protective titer against a given pathogen is known, then protection against a pathogen also means that a statistically significant percentage of fish reached protective titer upon vaccination as described herein.

The term “total dose” refers to the sum of the doses of the antigen administered with each member of the plurality of injections. For example and without limitations, if the vaccine is administered in three injections, wherein each injection contains 100 ng of the vector containing the nucleotide sequence encoding the antigen, then the total dose is 300 ng.

The expression that “the total dose is substantially equal to the effective dose” refers to the total dose which is at least 90% and no greater than 100% of the effective dose. Conversely, the expression that “the total dose is less than the effective dose” refers to the total dose which is less than 90% of the effective dose.

The term “the same protective immune response” refers to a response which is at least 95% and no greater than 110% of the reference value, which is the immune response elicited by a single injection containing the dose of the antigen equal to the total dose. Conversely, the term “greater protective immune response” refers to a response which is greater than 110% of the reference value unless the tissue damage is measured, in which case the term “greater protective immune response” refers to a response which is less than 95% of the reference value.

It is believed that administration of the same amount of the vaccine in several injections is likely to improve the antigen uptake by the host cells thus leading to increased antigen expression and allowing antigen dose sparing to achieve substantially the same protective immune response as a greater antigen dose administered in a single injection (i.e., the effective dose), and/or allowing an improved protective immune response if the total dose of the vaccine administered in several injections is the same dose as the effective dose.

In a first broad aspect, the invention provides a vaccine comprising a nucleotide sequence encoding an antigen derived from a pathogen, for use in a method of protecting fish from infection from said pathogen, said method comprising administering said vaccine to said fish intramuscularly in a plurality of injections, wherein the members of the plurality of injections are delivered into different injection sites; each member of said plurality contains less than an effective dose of said vaccine; the total dose of said vaccine is no greater than the effective dose; and all members of the plurality are delivered to said fish substantially simultaneously.

In a set of preferred embodiments, said administration of the members of the plurality of the injections provides a synergistic protective immune response, which is higher than a protective immune response elicited by the same dose of the same vaccine administered in a single effective dose, wherein both groups of fish (the group treated with a single effective dose and the group treated according to the claims of this disclosure) are kept under the same conditions. This may be important because fish are poikilotherms and the tank water temperature may be important. The temperature itself is not as important as keeping the two groups under the same conditions.

Also disclosed is a vaccine administered according to the method disclosed above, wherein the total dose of said vaccine is less than the effective dose.

The invention also provides a vaccine comprising a nucleic acid sequence encoding an antigen derived from a pathogen affecting fish, for use in a method to actively protect fish against the pathogen, said method comprising administering said vaccine to said fish intramuscularly in a plurality of injections, wherein the members of the plurality are delivered into different injection sites; all members of the plurality are delivered to said fish substantially simultaneously, and wherein the members of the plurality act synergistically or wherein the total dose administered in the plurality of the injections provides a faster onset of immunity than the same dose administered in a single injection of the same formulation, when administered to fish kept under the same conditions. Preferably, the members of the plurality act synergistically and the total dose administered in the plurality of the injections provides a faster onset of immunity than the same dose administered in a single injection of the same formulation, when administered to fish kept under the same conditions.

The immune response may be measured in multiple ways, depending on the pathogen and knowledge in the art. For example, one may determine the titer of the antibody specific to the pathogen. In other embodiments, the protective immune response may be measured by the percentage of seroconverted animals. This endpoint is particularly suitable for the pathogen where the protective titer has been established.

In other embodiments, mortality rate or survival rate may be a proper endpoint for determining the synergistic effect. Viral count may also be a suitable endpoint for determining the synergistic effect. Alternatively, prevalence of infection (i.e., the ratio of virus-positive fish to total fish) in at least organ at one or more time points may be a suitable endpoint. The immune response may also be measured by organ or tissue damage (or rather, the lack of the organ or tissue damage) in response to a challenge. Synergy in least one of these endpoints is indicative of synergistic protective immune response.

The vaccines administered according to the methods may provide an earlier onset of immunity than the same vaccines administered in a single injection, when administered to fish kept under the same conditions. The onset of immunity may be measured by the same methods as the immune response, including without limitations, viral counts, tissue damage, or percentage of fish that does not exhibit viral infection. An earlier post-challenge immune response in the group administered a plurality of injections compared to the group administered a single injection indicates an earlier onset of immunity, even if at the later stage, the respective immune response elicited by the single injection and the multiple injections may be less pronounced.

Vaccines suitable for the use according to the method described herein are vaccine where the nucleic acid molecule encoding the antigen enters the cells of the host and express the antigen. Such vaccine may be DNA vaccines or mRNA vaccines. Both types of such vaccines have been known in the art.

mRNA based vaccines against COVID-19 have been approved for human use. Thus, in certain embodiments, the vaccine comprises a mRNA sequence of a protein derived from a pathogen that causes fish disease.

DNA vaccines have also been known. At least one DNA vaccine, CLYNAV®, has been approved in Europe for use in protection of salmon from pancreatic disease caused by Salmonid Alphavirus. CLYNAV® consists of a DNA plasmid (pUK-SPDV-poly2 #1) dissolved in phosphate buffered saline. There is no adjuvant or preservative. The quantitative and qualitative composition has been adequately defined as ‘pUK-SPDV-poly2 #1 DNA plasmid coding for salmon pancreas disease virus (SPDV) proteins’, 5.1-9.4 μg/0.05 ml dose (101.6-188.4 μg/ml).

Thus, in the DNA vaccines according to different embodiments of the invention the nucleic acid sequence encoding the antigen is in a heterologous vector. Multiple heterologous vectors are suitable for the invention and are known in the art. Without limitations, the vectors include viral vectors, plasmid vectors, and circular DNA vectors, also known as doggybone vectors.

Suitable heterologous viral vectors include, without limitations, alphaviruses such as SAV, rhabdoviruses such as VHSV and IHNV, paramyxoviruses such as ASP, adenoviruses, poxviruses such as Salmon gill poxvirus, and the like. These viruses can be genetically modified to remove the parts of the viral genomes responsible for replication. Thus, the resulting viruses would be infectious to fish cells and suitable for production of the antigen, but not be pathogenic.

Suitable plasmids include, without limitations pUC-based vectors, pVAX-vectors, pcDNA-vectors, NTC-vectors. In a set of preferred embodiments, the vector is NTC9385R (Nature Technology Corporation) or a variant thereof.

In other embodiments, a relatively new “doggybone” or DBDNA™ plasmid may be used as a vector. DBDNA™ plasmids as well as the process of making these plasmids have been described at least in WO2018033730, WO2016034849, WO2019193361, WO2012017210 and WO2021161051.

The advantage of this approach is that the vector can be synthesized in a cell-free process thus improving manufacturing efficiency. The cell-free process preferably involves amplification of the template via strand displacement replication. This synthesis releases a single stranded DNA, which may in turn be copied into double stranded-DNA, using a polymerase. Alternatively, strand displacement can be achieved by supplying a DNA polymerase and a separate helicase. Replicative helicases may open the duplex DNA and facilitate the advancement of the leading-strand polymerase. The resulting double-stranded DNA concatemer is enzymatically cut and ligated thus forming the doggybone-like shape DNA construct. The doggybone vectors consist of telomerase recognition site, the desired construct (including the sequence encoding the antigen, the promoter, and the polyA site) and a sequence complementary thereto. Thus, the complementary portions of the vector hybridize to each other and form a helix.

Multiple pathogens are suitable for the use as the sources of the antigen in the nucleic acid-based vaccines disclosed herein. In certain embodiments, the pathogen is a virus. Suitable viruses may be selected from the group consisting of salmonid alphavirus (SAV), viral hemorrhagic septicemia virus (VHSV) Infectious hematopoietic necrosis virus (INHV), infectious pancreatic necrosis virus (IPNV), Infectious salmon anaemia (ISA) virus (ISAV), Piscine Myocarditis virus (PMCV), Piscine Orthoreovirus (PRV), Lumpfish virus, Viral Nervous Necrosis Virus (NNV), Infectious Spleen and Kidney Necrosis Virus (ISKNV), and Tilapia Lake Virus. In a particular embodiment, the virus is salmonid alphavirus. In another embodiment, the virus is PMCV.

In other embodiments, the pathogen is a bacterium. Suitable bacteria include without limitations Piscirickettsia sp. Aeromonas sp., Vibrio sp., Listonella sp., Moritella viscosa, Photobacterium damselae, Flavobacterium sp., Yersinia sp., Renibacterium sp., Streptococcus sp., Lactococcus sp., Leuconostoc sp., Bifidobacterium sp., Pediococcus sp., Brevibacterium sp., Edwarsiella sp., Francisella sp., Pseudomonas sp., Cytophaga sp., Nocardia sp., and Mycobacerium sp.

Particularly preferred are antigens from intracellular bacterial pathogens including, without limitations, Piscirickettsia sp., Edwardsiella sp., Yersinia sp., Francisella sp., Photobacterium sp., Mycobacterium sp., and Renibacterium sp.

Surface proteins from the viruses and bacteria may be suitable candidates for the nucleic-acid-based vaccines disclosed herein.

In other embodiments, the pathogen is a parasite, such as, for example, sea lice. In certain embodiments, the sea lice are selected from the genus Lepeophtheirus or Caligus, and the antigen may be a midgut protein or an immunogenic fragment thereof. See, e.g., U.S. Pat. No. 11,167,017.

Certain vaccines, particularly suitable for administration to salmonids, such as Salmo salar, comprise nucleic acid sequences that encode immunogens that elicit protective immune response against salmonid alphavirus. Suitable non-limiting examples of such antigens include, without limitations SEQ ID NO: 1 or SEQ ID NO: 2.

Other vaccines also suitable for administration to salmonids, such as Salmo salar, comprise nucleic acid sequences that encode immunogens that elicit protective immune response against piscine myocarditis virus (PMCV). Suitable non-limiting example of such an antigen is a protein encoded by ORF-1 of PMCV (e.g., SEQ ID NO: 3), or a fragment thereof, including, without limitations SEQ ID NO: 4.

Yet other vaccines suitable for administration to such as Salmo salar, comprise nucleic acid sequences that encode immunogens that elicit protective immune response against piscine myocarditis virus (PMCV) and immunogens that elicit protective immune response against salmonid alphavirus, as described above.

In other embodiments, the pathogen is a parasite such as, for example, a sea louse, particularly of genus Lepeophtheirus, and more particularly, of species Lepeophtheirus salmonis. Peptides from sea louse have been suggested as potential antigens in a vaccine. Some suitable candidates are disclosed in the U.S. Pat. No. 11,167,017.

The nucleic acid sequence in the DNA vaccine is generally under the control of a suitable promoter. Promoters suitable for the vaccine according to the invention should be able to initiate transcription in the host organism. In the embodiments where the host is a salmonid, such as Salmo salar, suitable promoters include, without limitations, simian virus 40 early promoter (SV40), cytomegalovirus immediate-early promoter (CMV), human Ubiquitin C promoter (UBC), human elongation factor 1a promoter (EF1A), mouse phosphoglycerate kinase 1 promoter (PGK), and chicken β-Actin promoter coupled with CMV early enhancer (CAGG).

The vector may further comprise a nucleic acid sequence encoding a molecular immunomodulator. Suitable molecular immunomodulators include interferons. It has been previously demonstrated that nucleic acid sequences encoding salmon interferons delivered in a DNA vaccine enhance the antigen-specific immune response. Accordingly, in certain embodiments, the molecular immunomodulator is an interferon selected from the group consisting of salmon IFNa, IFNb, IFNb1, IFNa2, and IFNc.

The vaccine may also comprise an adjuvant. Suitable adjuvants include, without limitations, saponins (e.g., Quil A), alum, CpG oligonucleotides, oligoribonucleotides, cytokines, glycolipids such as BAY®1005, quaternary amines such as dimethyl dioctadecyl ammonium bromide (hereinafter, “DDA”). Complexes comprising the saponin, the sterol (e.g., cholesterol), and, optionally, a phospholipid, have been described in the art. Combinations of CpG oligonucleotides and Saponin, CpG and cholesterol, and CpG and alum have been reported to elicit synergistic effects.

In certain embodiments, the vaccine may include liposomal adjuvant and/or carrier to facilitate the transport of the vector across the cell membrane and thus result in an increased expression of the antigen and/or the molecular immunomodulator. A suitable non-limiting example of such liposomal adjuvant/carrier system is described, for example, in the U.S. Pat. No. 10,456,459.

The vaccine according to the invention may further comprise excipients such as preservatives, stabilizers, buffers and the like.

Multiple fish species are suitable for the vaccination according to the method described herein. Suitable species include salmonids (including the species of genus Salmo and genus Oncorhynchus) as well as sea bass (Dicentrarchus labrax), as well as warm-water fish including Tilapia (Oreochromis niloticus) and Pangasius (Pangasius Hypophthalmus). Other species include white fish, Arctic charr, Mandarin perch and largemouth bass. The fish can be vaccinated according to the methods disclosed herein when the fish weighs between about 15 grams and about 200 grams, more preferably, between about 40 and about 110 grams.

As mentioned above, the vaccine according to the invention is delivered in several portions via a plurality of injections administered substantially simultaneously (the first portion and the last portion are administered within five minutes). Preferably, the last portion is administered no later than 4 minutes, no later than 3 minutes, no later than 2 minutes, no later than 1 minute, no later than 45 seconds, no later than 30 second, and no later than 15 seconds, and no later than 5 seconds, and no later than 1 second from the first portion of the vaccine.

The exact number of the members of the plurality of injections would generally be 2, 3, 4, or 5, more preferably, 2 or 3.

The volumes of the members of the plurality may be determined by a person of the ordinary skill in the art, but in general, the volumes may be independently chosen from 0.01 ml to about 0.25 ml, and may include about 0.02 ml, about 0.025 ml, about 0.05 ml, about 0.075 ml, about 0.1 ml, about 0.15 ml, about 0.2 ml, or 0.25 ml. It is currently preferred that the members of the plurality of injections should contain substantially the same amount of the antigen.

Multiple methods exist to administer the injections substantially simultaneously. In certain embodiments, commercially available fish vaccination equipment may be fitted with a multi-needle injection tip, where the tips of the needles are configured to be at a desired distance from each other. Suitable vaccination machines include NFTT lines of products (Pharmaq) and the specific models include NFT 20, NFT 25 and NFT 30. NFT 20, NFT 25 deliver vaccines intraperitoneally but can be reconfigured for intramuscular injections also. NFT 30 NFT 30 has a special DNA module that is capable of delivering a DNA vaccine intramuscularly, into the filet of the fish.

The machines handle fish in the sizes from 120 mm to 250 mm (20-150 grams). Once finished, it sorts the vaccinated fish into three different sizes. In addition, it has a channel for misplaced, undersized or rejected fish.

On the other end of the spectrum, the vaccine may be administered manually via a syringe containing one or several needles. See, e.g., MICRO-MATIC® syringe sold by Pharmaq. This product comes in single and double size. The single syringe is available in two sizes: 0.05 ml per dose and 0.1 ml per dose. Additionally, the 0.05 ml syringe can be supplied with an interchangeable 0.025 ml piston if required. The double syringe is available in three different dose size combinations: 0.05 ml+0.05 ml per dose; 0.05 ml+0.1 ml per dose; and 0.1 ml+0.1 ml per dose. The 0.05 ml syringe can be supplied with an interchangeable 0.025 ml piston if required. These syringes can be used for both water-based and oil-based vaccine formulations. The dose size can easily be adjusted +/−10%. The dose accuracy is documented to deviate less than 3%.

The pressure to deliver the injection is not important and may be derived from hydraulic, pneumatic, electrical, or mechanical sources.

Generally, the exact number of the injections (i.e., the members of the plurality of injections) may be determined experimentally. Preferably, the number is between two and five, more preferably, between two and four, and most preferably, two or three.

In one embodiment, there are three members of the plurality of injections and the antigen comprises a PMCV antigen, such as for example SEQ ID NO: 3 or SEQ ID NO: 4. In these embodiments, the vaccine may also contain an immunomodulator such as interferon. Preferably, the interferon is IFNb. In another embodiment, there are two members of the plurality of injections and the antigen comprises a PMCV antigen, such as for example SEQ ID NO: 3 or SEQ ID NO: 4. In these embodiments, the vaccine may also contain an immunomodulator such as interferon. Preferably, the interferon is IFNb.

In another embodiment, there are two members of the plurality of injection and the antigen comprises a SAV antigen, such as for example SEQ ID NO: 1 or SEQ ID NO 2.

The following examples are presented as illustrative embodiments, but should not be taken as limiting the scope of the invention. Many changes, variations, modifications, and other uses and applications of this invention will be apparent to those skilled in the art.

### EXAMPLES

### Example 1. Multiple Injections of SAV DNA Vaccine Result in Lower Viral Loads and Lower Clinical Scores than a Single Injection

Materials and Methods: Atlantic salmon parr weighing 26 grams in average were vaccinated in freshwater by intramuscular vaccination of a standard dose of a PD DNA vaccine. For one group, the entire 0.05 ml dose was administered in a single injection. For the other group, the 0.05 mL dose was administered in 2 consecutive injections of 0.025 mL each. Fish injected with phosphate buffered saline (PBS) served as negative controls. The fish were kept in the same tank throughout the study. Starting from the day after vaccination, the fish were exposed to continuous light to induce smoltification, inducing the physiological changes required to prepare the fish for transfer to seawater. After an immunization period of 6 weeks (42 days, 500 degree days), the fish were transferred to sea water and exposed to SAV3 by cohabitation challenge. Challenge was performed by intraperitoneal injection of naïve fish with infectious SAV3 material, which were introduced to the same tank to expose vaccinated and control fish to SAV3 in a way mimicking a natural outbreak of infection. 29-30 fish were challenged per group for the vaccinated fish, and 20 fish were challenged for the negative control fish. All surviving fish were sampled at termination 5 weeks (36 days) post onset of cohabitation challenge. The fish were weighed, and heart and pancreas samples were obtained on formalin and RNALATER® (both of which are important target organs for PD). As no mortalities were obtained during the observation period for the two vaccinated groups, and only a single mortality (5%) was obtained for the negative control fish, efficacy was primarily evaluated by evaluation of weight, severity of tissue damage to the heart and pancreas and quantity of SPDV in the heart and pancreas.

Statistical analysis of cardiac viral load and pancreas viral load for vaccinated groups compared to the negative control group was performed by Mann-Whitney test using GRAPHPAD PRISM @ v.8.1.1. Statistical analysis of weight of vaccinated groups at termination of the challenge period compared to the negative control group was performed by un-paired t-test with Welch's correction, using GRAPHPAD PRISM @ v.8.1.1.

Quantification of SPDV in heart and pancreas tissue samples was performed by a probe-based reverse transcriptase quantitative PCR (RT-qPCR) assay. The assay is targeting the non-structural protein 1 (nsP1) of SPDV and is capable of detecting all known salmonid alphaviruses (Hodneland & Endresen, 2006). The analyses were performed by an officially accredited diagnostic lab (PHARMAQ Analytiq), authorized for official verification of SPDV infection in Salmonid fish in Norway.

Severity of tissue damage for surviving fish at termination 5 weeks post-challenge (wpc) was analysed by histopathology on a scale of 0-3 (pancreas) and 0-4 (heart) using the scoring system described in Graham et al, Journal of fish diseases, vol 34, issue 4, 237-286. 2011. This scoring system is provided in Table 1.

TABLE 1
Tissue damage scoring table

| | | |
|---|---|---|
| Pancreas | 0 | Normal appearance |
| | 1 | Focal pancreatic acinar cell necrosis |
| | 2 | Significant multifocal necrosis/atrophy of pancreatic acinar |
| | | tissue, plus some remnants remaining |
| | 3 | Total absence of pancreatic acinar tissue |
| | R | Recovery pancreas |
| Heart | 0 | Normal appearance |
| | 1 | Focal myocardial degeneration ± inflammation (<7 fibres |
| | | affected) |
| | 2 | Focal myocardial degeneration ± inflammation (<15% of |
| | | heart affected) |
| | 3 | Multifocal myocardial degeneration ± inflammation |
| | | (>15 & <50% heart affected) |
| | 4 | Severe diffuse myocardial degeneration ± inflammation |
| | | (>50% heart affected) |
| | R | Repair |
| Red & white skeletal | 0 | Normal appearance |
| muscle | 1 | Focal myocytic degeneration ± inflammation |
| | 2 | Multifocal myocytic degeneration ± inflammation |
| | 3 | Severe diffuse myocytic degeneration ± inflammation |
| | R | Repair |

The results are in Tables 2 (viral counts), 3 (severity of tissue damage) and 4 (weight).

TABLE 2
PCR results for surviving fish at termination 5 wpc.

| | | | PD DNA | Negative |
|---|---|---|---|---|
| | | PD DNA vaccine | vaccine 2 × | control |
| | | 1 × 0.05 mL | 0.025 mL | (PBS) |
| Heart | Prevalence | 17.2% | 6.7% | 84.2% |
| | | (5/29 fish) | (2/30 fish) | (16/19 fish) |
| | Geometric | 32.5 | 33.3 | 30.2 |
| | mean Ct | | | |
| | p-value | <0.0001 | <0.0001 | Not relevant |
| | against PBS | | | |
| Pancreas | Prevalence | 27.6% | 13.3% | 63.2% |
| | | (8/29 fish) | (4/30 fish) | (12/19 fish) |
| | Geometric | 31.1 | 32.9 | 31.9 |
| | mean Ct | | | |
| | p-value | 0.1687 | 0.0001 | Not relevant |
| | against PBS | | | |

The PCR results demonstrate that both single and double injections resulted in reduced prevalence of infection and lower viral load (higher count), but the prevalence was lower in the double-injection group. The heart viral counts in both the single-injection group and the double-injection group were significantly different from control. The pancreas viral counts were significantly different from the control in the double-injection group. These results demonstrate that double-injection with 2×0.025 ml of the vaccine is more effective than a single injection with 0.05 ml of the vaccine.

TABLE 3
Severity of tissue damage for surviving fish at termination 5 wpc

| | | PD DNA | PD DNA | Negative |
|---|---|---|---|---|
| | | vaccine | vaccine 2 × | control |
| | | 1 × 0.05 mL | 0.025 mL | (PBS) |
| Heart | Mean score | 0.41 | 0.13 | 2.21 |
| | Range | 0-2 | 0-1 | 2-3 |
| | (min-max) | | | |
| | p-value | <0.0001 | <0.0001 | Not relevant |
| | against PBS | | | |
| Pancreas | Mean score | 0.31 | 0.17 | 2.16 |
| | Range | 0-2 | 0-2 | 0-3 |
| | (min-max) | | | |
| | p-value | <0.0001 | <0.0001 | Not relevant |
| | against PBS | | | |

Tissue damages in the pancreas and the heart of the fish in the single-injection group and in the double-injection group were different from the damages in the pancreas and the hearts, respectively, of the control group. Notably, in both pancreas and the hearts, the double-injection group tended to show smaller damages than the single injection group (0.13 vs 0.41 in the heart and 0.17 vs 0.31 in the pancreas).

TABLE 4
Weight of surviving fish at the time of
challenge and at termination 5 wpc.

| | PD DNA | PD DNA | Negative |
|---|---|---|---|
| | vaccine | vaccine | control |
| | 1 × 0.05 mL | 2 × 0.025 mL | (PBS) |
| Mean weight at | 38.8 | 39.1 | 40.5 |
| challenge | | | |
| Mean weight at | 53.7 | 51.3 | 43.7 |
| termination | | | |
| Mean % growth | 38.5% | 31.3% | 8.2% |
| p-value against PBS | <0.0001 | 0.0002 | Not relevant |

The weight of the vaccinated groups 5 weeks post onset of cohabitation challenge demonstrates that both vaccinated groups were highly protected against PD-related growth reduction, with notably higher weight and growth rate compared to the negative control group. No statistically significant differences have been observed between the one-injection and two-injection groups.

The increase of the number of injections from one to two resulted in lower viral load and the decreased viral prevalence in both heart and pancreas at five weeks post challenge. Prevalence in both heart and pancreas decreased more than two-fold in the double-injections group compared to a single-injection group even though the total amount of the vaccine was the same in both groups

### Example 2. Multiple Injections of PMCV DNA Vaccine Result in Lower Viral Load and Lower Histopathology Scores than a Single Injection

Atlantic salmon (n=150) with mean weight of 29 g were kept in a 500L tank with freshwater (12C) with a 12:12 light: dark light-regime. The fish were starved for one day and then anaesthetized using MS222 (Tricaine, PHARMAQ AS), tagged into one of five groups (n=30 per group) by shortening of adipose fin or maxillae and vaccinated intramuscularly with a DNA-plasmid expressing a partial ORF1 PMCV antigen and salmon IFNb as a molecular adjuvant. One of the five groups was given a negative control vaccine (PBS) while the remaining four groups were injected 1, 2, 3 or 4 times under the same anaesthetic period with vaccines containing the PMCV ORF1 encoding sequence. The concentration of plasmid was adjusted for each group, so that the total amount of plasmid was 10 μg per fish, regardless of number of injections. Table 5 details the groups at vaccination.

TABLE 5
Experimental setup

| | | Number of | | | |
|---|---|---|---|---|---|
| | | injections per | Concentration of | Total amount of DNA | Fish |
| Group | Tag | fish | plasmid (ug/ml) | plasmid (ug/fish) | (n) |
| 1 | None | 1 × 0.05 ml | 200 | 10 | 30 |
| 2 | Adipose fin | 2 × 0.05 ml | 100 | 10 | 30 |
| 3 | Right Maxillae | 3 × 0.05 ml | 67 | 10 | 30 |
| 4 | Left Maxillae | 4 × 0.05 ml | 50 | 10 | 30 |
| 5 | Right Maxillae + | 4 × 0.05 ml | 0 (PBS) | 0 | 30 |
| | Adipose fin | | | | |

Following 7 weeks of immunization, all fish were anaesthetized again and injected intraperitoneally with 0.1 ml of a kidney homogenate containing infectious PMCV particles. Hearts were then sampled 3 and 7 weeks after challenge from 15 fish per group per time-point. The hearts were stored on RNALATER® and sent to the diagnostic laboratory PHARMAQ Analytiq (Bergen, Norway) for real-time PCR detection of PMCV RNA. Hearts sampled 7 weeks post challenge were also examined for heart pathology (PHARMAQ Analytiq, Bergen Norway). The viral counts three and seven weeks post-challenge are summarized in tables 6 and 7, respectively. Histological analysis of heart atriums is summarized in table 8.

TABLE 6
Real-time PCR results from hearts of fish
challenged with PMCV 3 weeks earlier.
Negative fish were given a Ct-value of 40 by default.

| | | | Number of | |
|---|---|---|---|---|
| | Number of | | PMCV | Mean ct |
| | injections | | positive | value |
| Group | per fish | Fish (n) | fish | (PMCV) |
| 1 | 1 × 0.05 ml | 15 | 13 | 33, 37 |
| 2 | 2 × 0.05 ml | 15 | 10 | 35, 38 |
| 3 | 3 × 0.05 ml | 15 | 7 | 37, 51 |
| 4 | 4 × 0.05 ml | 15 | 10 | 36, 23 |
| 5 | 4 × 0.05 ml | 15 | 15 | 32, 03 |

TABLE 7
Real-time PCR results from hearts of fish
challenged with PMCV 7 weeks earlier.
Negative fish were given a Ct-value of 40 by default.

| | | | Number of | |
|---|---|---|---|---|
| | Number of | | PMCV | Mean ct |
| | injections | | positive | value |
| Group | per fish | Fish (n) | fish | (PMCV) |
| 1 | 1 × 0.05 ml | 15 | 10 | 33, 74 |
| 2 | 2 × 0.05 ml | 15 | 10 | 35, 34 |
| 3 | 3 × 0.05 ml | 15 | 8 | 37, 26 |
| 4 | 4 × 0.05 ml | 15 | 10 | 35, 59 |
| 5 | 4 × 0.05 ml | 15 | 15 | 27, 22 |

TABLE 8
Histological analysis of heart atriums. Histological lesions
were scored from 0 (normal) to 4 (severe pathology). The number
of fish given each score for each group is indicated.

| | Number of | Mean | Distribution of scores | | | | |
|---|---|---|---|---|---|---|---|
| Group | injections per fish | score | 0 | 1 | 2 | 3 | 4 |
| 1 | 1 × 0.05 ml | 0.73 | 5 | 4 | 2 | 0 | 0 |
| 2 | 2 × 0.05 ml | 0.71 | 8 | 4 | 1 | 0 | 1 |
| 3 | 3 × 0.05 ml | 0.53 | 8 | 6 | 1 | 0 | 0 |
| 4 | 4 × 0.05 ml | 0.86 | 5 | 7 | 1 | 1 | 0 |
| 5 | 4 × 0.05 ml | 2.40 | 0 | 0 | 9 | 6 | 0 |

The increase of the number of injections from 1 to three resulted in both the increased viral counts (lower viral load) and the percentage of fish that were found negative (from two in the group treated with one injection to eight in the group treated with three injections) at three weeks post challenge. At seven weeks post-challenge, the number of negative fish in singe-, double- and quadruple-injection group was 5 out of 15, and in the triple-injection group, the number of negative fish was 7 out of 15. These results suggest that the vaccine administered in multiple injections facilitates a faster onset of immunity. No significant weight differences were found between one-, two-, three-, and four-injection groups.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1-29. (canceled)

30. A method of administering a total dose of a nucleic-acid-based vaccine to a fish, the method comprising delivering a plurality of portions of said total dose into different injection sites of said fish, wherein said plurality of portions is injected substantially simultaneously, wherein the members of the plurality act synergistically or wherein the total dose administered in the plurality of the injections provides a faster onset of immunity than the same dose administered in a single injection of the same formulation, when administered to fish kept under the same conditions.

31. The method of claim 31, wherein the members of the plurality act synergistically or wherein the total dose administered in the plurality of the injections provides a faster onset of immunity than the same dose administered in a single injection of the same formulation, when administered to fish kept under the same conditions.

32. The method of claim 30, wherein the total dose is smaller than an effective dose of the same vaccine.

33. The method according to claim 30 wherein the plurality has 2, 3, or 4 members.

34. The method according to claim 30, wherein said plurality of portions is delivered within 1 minute.

35. (canceled)

36. (canceled)

37. The method according to claim 30, wherein each member of said plurality contains substantially the same amount of the antigen.

38. The method according to claim 37, wherein
a. the plurality has two members and each member of said plurality has about 1/2 of the total dose of the antigen; or
b. the plurality has three members and each member of said plurality has about 1/3 of the total dose of the antigen.

39. The method according to claim 30, wherein said pathogen is selected from the group consisting of salmonid alphavirus (SAV), viral hemorrhagic septicemia virus (VHSV) Infectious hematopoietic necrosis virus (INHV), infectious pancreatic necrosis virus (IPNV), Infectious salmon anaemia (ISA) virus (ISAV), Piscine Myocarditis virus (PMCV), Piscine Orthoreovirus (PRV), Lumpfish virus, Viral Nervous Necrosis Virus (NNV), Infectious Spleen and Kidney Necrosis Virus (ISKNV), Tilapia Lake Virus, Piscirickettsia sp., Edwardsiella sp., Yersinia sp., Francisella sp., Photobacterium sp., Mycobacterium sp., Renibacterium sp., Lepeophtheirus sp. and Caligus sp.

40. The method according to claim 30, wherein the fish is a salmonid and the pathogen is selected from the group consisting of salmonid alphavirus (SAV), viral hemorrhagic septicemia virus (VHSV) Infectious hematopoietic necrosis virus (INHV), infectious pancreatic necrosis virus (IPNV), Infectious salmon anaemia (ISA) virus (ISAV), Piscine Myocarditis virus (PMCV), and Piscine Orthoreovirus (PRV).

41. The method according to claim 39, wherein:
a) the plurality has two or three members and the pathogen is PMCV or
b) the plurality has two members and the pathogen is SAV.

42. (canceled)

43. The method according to claim 30, wherein said nucleic acid sequence encoding said antigen is delivered by a heterologous vector.

44. The method according to claim 43, wherein the heterologous vector is a plasmid vector or a viral vector.

45. The method according to claim 43, wherein the heterologous vector further comprises a nucleic acid sequence encoding a molecular immunomodulator.

46. The method according to claim 45, wherein the molecular immunomodulator is an interferon.

47. The method according to claim 30 further comprising an adjuvant.

48. The method according to claim 47, wherein the adjuvant is selected from the group consisting of MPLA, CpG-containing oligodeoxyribonucleotides, oligoribonucleotides, saponins, sterols, and cationic lipids.

49. The method according to claim 30, wherein said vaccine comprises a means for transporting said nucleic-acid-based vaccine across cell membrane.

50. The method according to claim 49, wherein said means comprise lipid coating.
